(19) United States
(12) Patent Application Publication
(10) Pub. No.: US 2017/0224311 A1
(43) Pub. Date: Aug. 10, 2017

(54) Control System and Method for Controlling an Ultrasonic Probe

(71) Applicant: National Kaohsiung University of Applied Sciences, Kaohsiung City (TW)

(72) Inventors: Jian-Chiun LIOU, Kaohsiung City (TW); Tien-Tsorng SHIH, Taipei City (TW)

(21) Appl. No.: 15/016,050

(22) Filed: Feb. 4, 2016

(51) Int. Cl. A61B 8/08; A61B 8/00

(52) U.S. Cl. CPC A61B 8/5215; A61B 8/4488; A61B 8/56

(57) ABSTRACT

A control system for controlling an ultrasonic probe includes a module for assigning address parameters respectively to ultrasonic transducers of the probe according to a physical position order of the ultrasonic transducers, a module for arranging the address parameters in an activation sequence, and a module for generating activating signals each corresponding to a respective address parameter. Sequential two address parameters in the activation sequence correspond to two ultrasonic transducers that are non-sequential to each other in the physical position order. Each activating signal is transmitted to a respective ultrasonic transducer that is assigned with the address parameter to which the activating signal corresponds.

### FIELD

The disclosure relates to a control system and method, and more particularly to a control system and method for controlling an ultrasonic probe.

### BACKGROUND

In a conventional medical ultrasonic probe, ultrasonic transducers operate to generate ultrasound waves. Due to the elasticity of piezo-electric polycrystalline ceramic material of which the ultrasonic transducers are made, the ultrasonic transducers continue to vibrate for a while after they have stopped operating. As a result, sequential two of the ultrasonic transducers will interfere with each other, and an ultrasonic image obtained thereby will be adversely affected. To alleviate the interference, the conventional medical ultrasonic probe has damping material with high damping coefficient attached to a back side of the ultrasonic transducers for absorbing the undesirable vibration of the ultrasonic transducers. Alternatively, the ultrasonic image may be improved by signal processing and/or image processing.

### SUMMARY

Therefore, an object of the disclosure is to provide a control system that can alleviate at least one of the drawbacks of the prior arts.

According to one aspect of the disclosure, the control system is for controlling an ultrasonic probe including a plurality of ultrasonic transducers that are physically arranged in a row. The control system includes a probe controller. The probe controller includes an address assigning module, a mode setting module and an activating control module. The address assigning module is configured to assign a plurality of address parameters respectively to the ultrasonic transducers in a sequential order corresponding to a physical position order of the ultrasonic transducers in the row. The mode setting module is configured to arrange the address parameters in an activation sequence, where sequential two of the address parameters in the activation sequence correspond to respective two of the ultrasonic transducers that are non-sequential to each other in the physical position order. The activating control module is configured to generate a plurality of activating signals corresponding respectively to the address parameters, and to transmit the activating signals to the ultrasonic probe one by one every predetermined duration according to the activation sequence. Each of the activating signals is transmitted to a respective one of the ultrasonic transducers that is assigned with one of the address parameters to which the activating signal corresponds so as to drive the respective one of the ultrasonic transducers to transmit and receive ultrasonic waves.

Another object of the present disclosure is to provide a method for controlling an ultrasonic probe including a plurality of ultrasonic transducers that are physically arranged in a row.

According to another aspect of the disclosure, the method includes the following steps of:

assigning a plurality of address parameters respectively to the ultrasonic transducers in a sequential order corresponding to a physical position order of the ultrasonic transducers in the row;

arranging the address parameters in an activation sequence, where sequential two of the address parameters in the activation sequence correspond to respective two of the ultrasonic transducers that are non-sequential to each other in the physical position order;

generating a plurality of activating signals corresponding respectively to the address parameters; and

transmitting the activating signals to the ultrasonic probe one by one every predetermined duration according to the activation sequence, each of the activating signals being transmitted to a respective one of the ultrasonic transducers that is assigned with one of the address parameters to which the activating signal corresponds so as to drive the respective one of the ultrasonic transducers to transmit and receive ultrasonic waves.

### BRIEF DESCRIPTION OF THE DRAWINGS

Other features and advantages of the disclosure will become apparent in the following detailed description of the embodiment with reference to the accompanying drawings, of which:

FIG. 1 is a perspective view of an embodiment of a control system for controlling an ultrasonic probe according to the disclosure;

FIG. 2 is a block diagram of the embodiment of the control system;

FIG. 3 is a flowchart of an embodiment of a method for controlling an ultrasonic probe according to the disclosure;

FIG. 4A is a plot for illustrating an output signal outputted by one ultrasonic transducer of a conventional medical ultrasonic probe; and

FIG. 4B is a plot for illustrating an output signal outputted by one ultrasonic transducer of the ultrasonic probe operating under control of the control system according to this disclosure.

### DETAILED DESCRIPTION

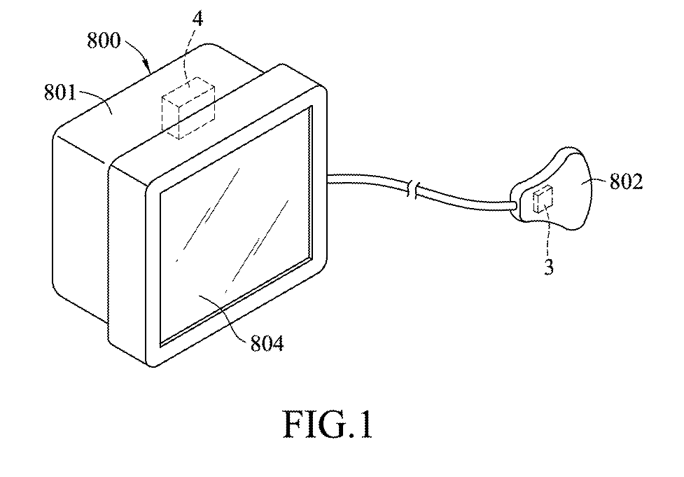

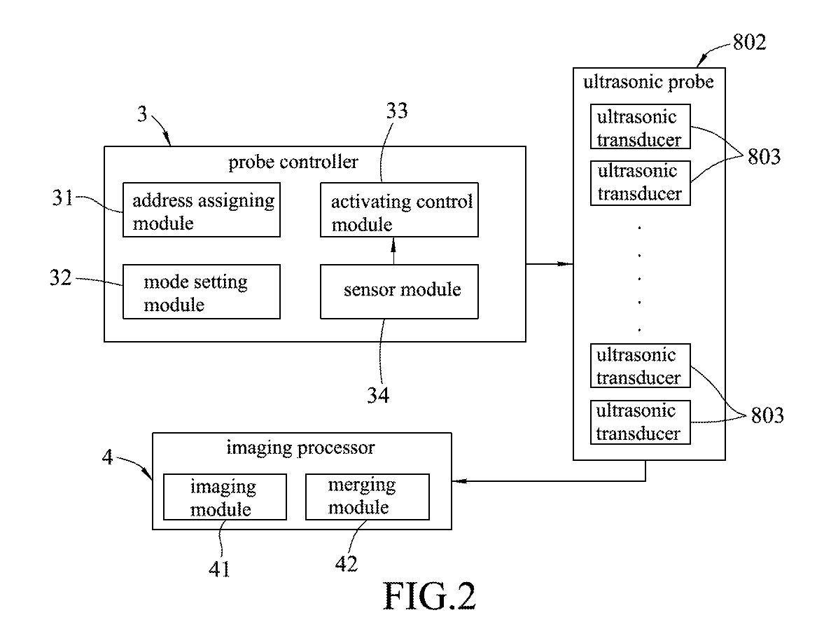

Referring to FIGS. 1 and 2, the embodiment of a control system according to this disclosure is applied to a medical ultrasound scanner 800 by one of software application, hardware circuit and a combination thereof. The medical ultrasound scanner 800 includes a console 801 having a touch screen 804 that is user-operable to control operation of the medical ultrasound scanner 800, and an ultrasonic probe 802 connected to the console 801. The ultrasonic probe 802 includes a plurality of ultrasonic transducers 803. The ultrasonic transducers 803 are physically arranged in an array (i.e., in a plurality of rows), and are configured to transmit and receive ultrasonic waves, to convert the ultrasound waves received thereby to respective output signals, and to output the output signals. In this embodiment, a number of the ultrasonic transducers 803 is 64. In other embodiments, the ultrasonic probe 802 may include 128 or 256 ultrasonic transducers 803. Since the medical ultrasound scanner 800 is an apparatus well known in the art and comes in various types, details and operation thereof will be omitted herein for the sake of brevity, and the implementation thereof is not limited to the type shown in FIG. 1.

The control system is for controlling the ultrasonic probe 802, and includes a probe controller 3 configured to control operation of the ultrasonic probe 802, and an imaging processor 4 configured to process the output signals received from the ultrasonic probe 802.

The probe controller 3 is disposed in the ultrasonic probe 802, and includes an address assigning module 31, a mode setting module 32, an activating control module 33 and a sensor module 34.

The address assigning module 31 is configured to assign a plurality of address parameters respectively to the ultrasonic transducers 803 in a sequential order corresponding to a physical position order of the ultrasonic transducers 803 in the array. The address parameters are integers, and are assigned respectively to the ultrasonic transducers 803 in numerical order corresponding to the physical position order of the ultrasonic transducers 803 in the array. For example, the address parameters are 1, 2, 3, 4, . . . , 64 in this embodiment.

The mode setting module 32 is configured to arrange the address parameters in an activation sequence, where sequential two of the address parameters in the activation sequence correspond to respective two of the ultrasonic transducers 803 that are non-sequential to each other in the physical position order. In detail, the mode setting module 32 is configured to operate in one of an odd-even mode and an arithmetic sequential mode with a common difference of N.

In the odd-even mode, the mode setting module 32 arranges one of odd ones and even ones of the address parameters in numerical order, followed by the other one of the odd ones and the even ones of the address parameters in numerical order to form the activation sequence. For example, the mode setting module 32 arranges the odd ones of the address parameters in numerical order, such as 1, 3, 5, 7, . . . , 63, followed by the even ones of the address parameters in numerical order, such as 2, 4, 6, . . . , 64, to form the activation sequence, 1, 3, 5, 7, . . . , 63, 2, 4, 6, . . . , 64. Alternatively, the activation sequence may be 2, 4, 6, . . . , 64, 1, 3, 5, 7, . . . , 63 in other embodiments.

In the arithmetic sequential mode with the common difference of N, the mode setting module 32 arranges the address parameters into a number (N) of finite arithmetic sequences, and strings the finite arithmetic sequences to form the activation sequence, where N is an integer greater than or equal to 2 and smaller than the number of the ultrasonic transducers 803 (i.e., 64 in this embodiment). Preferably, N is equal to or smaller than half of the number of the ultrasonic transducers 803. A difference between sequential two of the address parameters in each of the finite arithmetic sequences is equal to the common difference. For example, in a case where N=3, the mode setting module 32 arranges the address parameters into three arithmetic sequences, such as 1, 4, 7, 10, . . . , 64, and 2, 5, 8, 11, . . . , 62, and 3, 6, 9, 12, . . . , 63. Then, the mode setting module 32 strings these three arithmetic sequences to form the activation sequence, such as 1, 4, 7, 10, . . . , 64, 2, 5, 8, 11, . . . , 62, 3, 6, 9, 12, . . . , 63. In another case where N=4, the mode setting module 32 arranges the address parameters into four arithmetic sequences, such as 1, 5, 9, 13, . . . , 61, and 2, 6, 10, 14, . . . , 62, and 3, 7, 11, 15, . . . , 63, and 4, 8, 12, 16, . . . , 64. Then, the mode setting module 32 strings these four arithmetic sequences to form the activation sequence, such as 1, 5, 9, 13, . . . , 61, 2, 6, 10, 14, . . . , 62, 3, 7, 11, 15, . . . , 63, 4, 8, 12, 16, . . . , 64.

The activating control module 33 is configured to generate a plurality of activating signals corresponding respectively to the address parameters, and to transmit the activating signals to the ultrasonic probe 802 one by one every predetermined duration (for example, 1 μs) according to the activation sequence. Each of the activating signals is transmitted to a respective one of the ultrasonic transducers 803 that is assigned with one of the address parameters to which the activating signal corresponds, so as to drive the respective one of the ultrasonic transducers 803 to transmit and receive ultrasonic waves.

In the odd-even mode 321, the activating control module 33 activates the ultrasonic transducers 803 of the ultrasonic probe 802 in an order of physical positions of the ultrasonic transducers 803 in the array corresponding to the activation sequence 1, 3, 5, 7 . . . , 63, 2, 4, 6, 8, . . . , 64. Namely, the 1^{st}, 3^{rd}, 5^{th}, 7^{th}, and 63^{rd} ones of the ultrasonic transducers 803 in the array are sequentially activated, and then, the 2^{nd}, 4^{th}, 6^{th}, 8^{th}, . . . and 64^{th} ones of the ultrasonic transducers 803 in the array are sequentially activated. In the arithmetic sequential mode, the activating control module 33 activates the ultrasonic transducers 803 in an order of the physical position corresponding to the activation sequence 1, 4, 7, 10, . . . , 64, 2, 5, 8, 11, . . . , 62, 3, 6, 9, 12, . . . , 63 if N=3. Namely, the 1^{st}, 4^{th}, 7^{th}, 10^{th}, . . . and 64^{th} ones of the ultrasonic transducers 803 in the array are sequentially activated, and then, the 2^{nd}, 5^{th}, 8^{th}, 11^{th}, . . . and 62^{nd} ones of the ultrasonic transducers 803 in the array are sequentially activated, followed by the 3^{rd}, 6^{th}, 9^{th}, 12^{th}, . . . and 63^{rd} ones of the ultrasonic transducers 803 in the array. Accordingly, only one of the ultrasonic transducers 803 is activated every predetermined duration (1 μs), and sequential two of the ultrasonic transducers 803 in the physical position order in the array will not operate at the same time and will not interfere with each other.

The imaging processor 4 is disposed at the console 801, and is coupled to the ultrasonic transducers 803. The imaging processor 4 includes an imaging module 41 and a merging module 42. When the mode setting module 32 operates in the odd-even mode, the imaging module 41 is configured to receive the output signals from a first activated group of the ultrasonic transducers 803, which includes those ultrasonic transducers 803 activated by the activating signals corresponding to the odd ones of the address parameters, and to process the output signals from the first activated group to form a first partial-image. Similarly, the imaging module 41 is sequentially configured to receive the output signals from a second activated group of the ultrasonic transducers 803, which includes those ultrasonic transducers 803 activated by the activating signals corresponding to the even ones of the address parameters, and to process the output signals received from the second activated group to form a second partial-image. The merging module 42 is configured to merge the first and second partial-images to form a single ultrasonic image.

When the mode setting module 32 operates in the arithmetic sequential mode with the common difference of N, the imaging module 41 is configured to receive the output signals from each of N number of activated groups of the ultrasonic transducers 803, which include those ultrasonic transducers 803 driven by the activating signals transmitted according to a respective one of the finite arithmetic sequences, and to process the output signals received from each of the activated groups to form a respective partial-image. The merging module 42 is configured to merge the partial-images corresponding respectively to the multiple activated groups to form a single ultrasonic image.

The sensor module 34 is configured to detect whether the activating control module 33 sequentially transmits two or more of the activating signals respectively to sequential two or more of the ultrasonic transducers 803 in the physical position order.

When detecting that the activating control module 33 sequentially transmits two or more of the activating signals respectively to the sequential two or more of the ultrasonic transducers 803, the sensor module 34 makes the activating control module 33 inactivate the ultrasonic probe 802 so that interference among the ultrasonic transducers 803 can be avoided. The sensor module 34 further causes the console 801 to output a warning indication, for example, in the form of a warning light, a warning sound and/or a warning image.

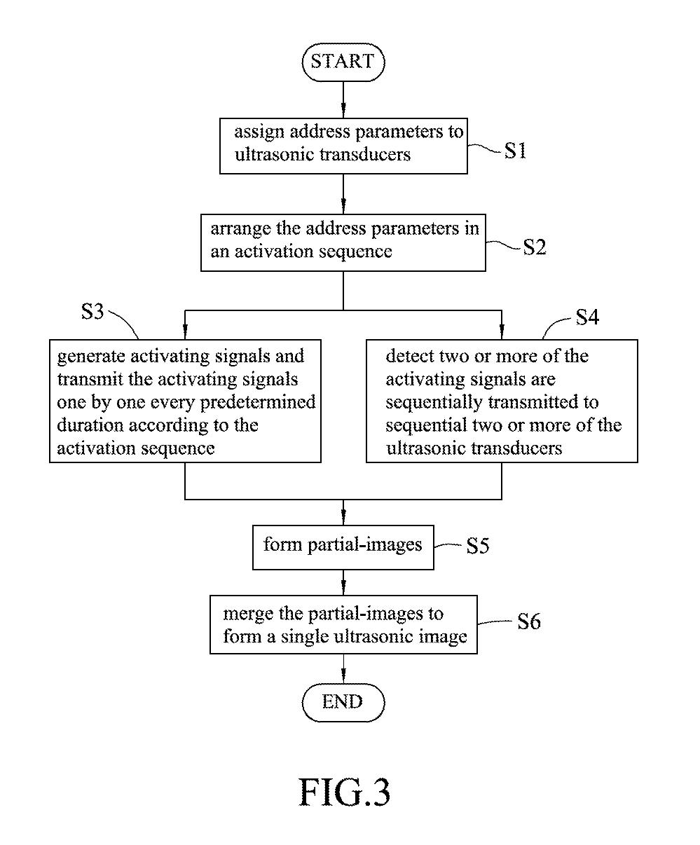

Referring to FIGS. 1, 2, and 3, a method for controlling the ultrasonic probe 802 is implemented by the control system, and includes the following steps.

In step S1, the address assigning module 31 assigns the address parameters respectively to the ultrasonic transducers 803 in the sequential order corresponding to the physical position order of the ultrasonic transducers 803 in the array.

In step S2, the mode setting module 32 arranges the address parameters in an activation sequence. In particular, the touch screen 804 displays a user interface (not shown) allowing a user to set operation of the mode setting module 32 in one of the odd-even mode and the arithmetic sequential mode. When the mode setting module 32 is set to operate in the arithmetic sequential mode, the user interface further allows the user to decide the value of the common difference, i.e., the value of N. Then, the mode setting module 32 generates the activation sequence in the above-mentioned manner according to a selected one of the odd-even mode and the arithmetic sequential mode 322.

In step S3, the activating control module 33 generates the activating signals corresponding respectively to the address parameters and transmits the activating signals to the ultrasonic probe 802 one by one every predetermined duration according to the activation sequence, so as to drive the respective ones of the ultrasonic transducers 803 to transmit and receive ultrasonic waves.

In step S4 simultaneous with step S3, the sensor module 34 detects whether the activating control module 33 sequentially transmits two or more of the activating signals to sequential two or more of the ultrasonic transducers 803 in the physical position order. For example, when the sensor module 34 detects that sequential two of the ultrasonic transducers 803 that are assigned with the address parameters 10 and 11 operate at the same time, the sensor module 34 makes the activating control module 33 inactivate the ultrasonic probe 802, and causes the console 801 to output the warning indication to notify the user of the need to check the medical ultrasound scanner 800.

In step S5, the imaging module 41 forms the partial-images. When the ultrasonic transducers 803 are activated in the activation sequence, each of the ultrasonic transducers 803 receives the ultrasound wave, converts the ultrasound wave into the output signal in a form of an electrical signal, and outputs the output signal. When the mode setting module 32 operates in the odd-even mode, the imaging module 41 receives the output signals from the first and second activated groups of the ultrasonic transducers 803, and processes the output signals to form two partial-images corresponding respectively to the first and second activated groups. When the mode setting module 32 operates in the arithmetic sequential mode with the common difference of N, the imaging module 41 receives the output signals from a number N of the activated groups of the ultrasonic transducers 803, and process the output signals to form a number N of the partial-images corresponding respectively to the number N of the activated groups.

In step S6, the merging module 42 merges the partial-images to form a single ultrasonic image, and enables the console 801 to display the ultrasonic image on the touch screen 804. Since there are many ways to merge a plurality of images into a single image, details of merging the partial-images will be omitted herein for the sake of brevity.

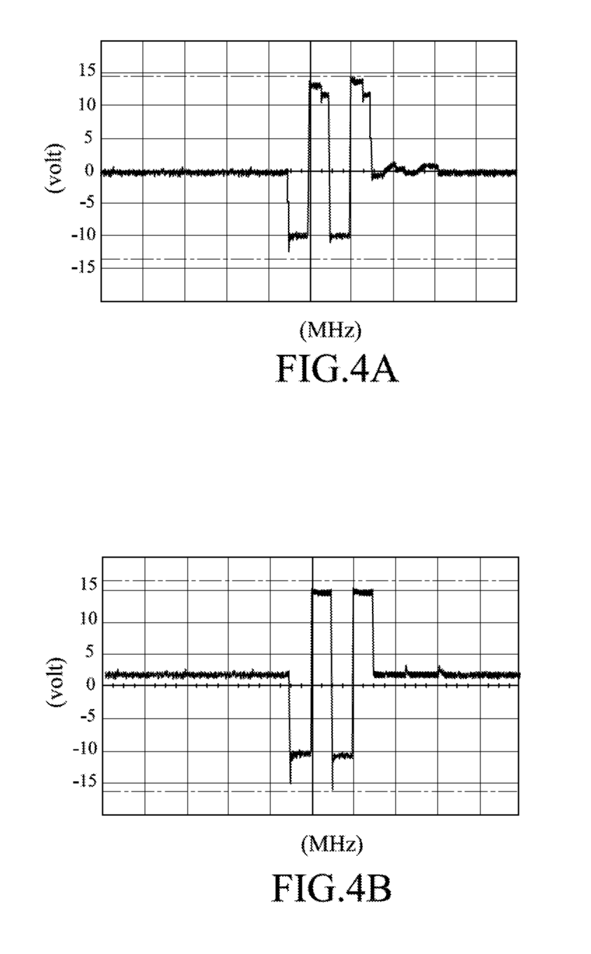

Referring to FIG. 4A, in a conventional medical ultrasonic probe, one ultrasonic transducer thereof will interfere with a sequential ultrasonic transducer that is sequential to said one ultrasonic transducer, so that the output signal outputted by the sequential ultrasonic transducer is unstable and has a peak-to-peak voltage level of 28.1 V in a frequency difference of 1 MHz. Referring to FIG. 4B, in the ultrasonic probe 802 controlled by the control system according to this disclosure, one of the ultrasonic transducers 803 will not interfere with a sequential one of the ultrasonic transducers 803 that is sequential in position to said one of the ultrasonic transducers 803, so that the output signal outputted by the sequential one of the ultrasonic transducers is relatively stable and has a peak-to-peak voltage level of 32.8 V in a frequency difference of 1 MHz.

In sum, the control system and method according to this disclosure control operation of the ultrasonic probe 802 in a manner that sequentially activated two of the ultrasonic transducers 803 are spaced apart by at least another one of the ultrasonic transducers 803 in the physical position order, such that interference among the ultrasonic transducers 803 is alleviated. Accordingly, quality of the partial-images is improved, and thus, quality of the resultant single ultrasonic image is also improved. In addition, since the output signals outputted by the ultrasonic transducers 803 have relatively great quality, complexity of back-end signal processing and image processing is significantly reduced. Moreover, when the sequential two or more of the ultrasonic transducers 803 in the physical position order are activated sequentially, the sensor module 34 makes the activating control module 33 inactivate the ultrasonic probe 802 and causes the console 801 to output the warning indication, such that the medical ultrasound scanner 800 will not operate in a conventional manner.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A control system for controlling an ultrasonic probe including a plurality of ultrasonic transducers that are physically arranged in a row, said control system comprising:
a probe controller that includes:
an address assigning module configured to assign a plurality of address parameters respectively to the ultrasonic transducers in a sequential order corresponding to a physical position order of the ultrasonic transducers in the row,
a mode setting module configured to arrange the address parameters in an activation sequence, where sequential two of the address parameters in the activation sequence correspond to respective two of the ultrasonic transducers that are non-sequential to each other in the physical position order, and
an activating control module configured to generate a plurality of activating signals corresponding respectively to the address parameters, and to transmit the activating signals to the ultrasonic probe one by one every predetermined duration according to the activation sequence, each of the activating signals being transmitted to a respective one of the ultrasonic transducers that is assigned with one of the address parameters to which the activating signal corresponds so as to drive the respective one of the ultrasonic transducers to transmit and receive ultrasonic waves.

2. The control system as claimed in claim 1, wherein the address parameters are integers, and are assigned respectively to the ultrasonic transducers in numerical order corresponding to the physical position order of the ultrasonic transducers in the row,
wherein said mode setting module is configured to operate in an odd-even mode, in which said mode setting module arranges one of odd ones and even ones of the address parameters in numerical order, followed by the other one of the odd ones and the even ones of the address parameters in numerical order to form the activation sequence.

3. The control system as claimed in claim 2, the ultrasonic transducers being configured to receive the ultrasound waves, respectively, and to output respective output signals,
wherein said control system further comprises an imaging processor that is configured to be coupled to the ultrasonic transducers, and that includes
an imaging module configured to
receive the output signals from a first activated group of the ultrasonic transducers, which include those of the ultrasonic transducers driven by the activating signals corresponding to the odd ones of the address parameters, and process the output signals received from the first activated group to form a first partial-image, and
receive the output signals from a second activated group of the ultrasonic transducers, which include those of the ultrasonic transducers driven by the activating signals corresponding to the even ones of the address parameters, and process the output signals received from the second activated group to forma second partial-image, and
a merging module configured to merge the first and second partial-images to form a single ultrasonic image.

4. The control system as claimed in claim 2, wherein said probe controller further includes a sensor module configured to detect whether said activating control module sequentially transmits two of the activating signals to sequential two of the ultrasonic transducers in the physical position order, and to make said activating control module inactivate the ultrasonic probe when detecting that said activating control module sequentially transmits two of the activating signals to the sequential two of the ultrasonic transducers.

5. The control system as claimed in claim 1, wherein the address parameters are integers, and are assigned respectively to the ultrasonic transducers in numerical order corresponding to the physical position order of the ultrasonic transducers in the row,
wherein said mode setting module is configured to operate in an arithmetic sequential mode with a common difference of N, in which said mode setting module arranges the address parameters into a number (N) of finite arithmetic sequences and strings the finite arithmetic sequences to form the activation sequence, where N is an integer greater than or equal to 2 and smaller than a number of the ultrasonic transducers, a difference between sequential two of the address parameters in each of the finite arithmetic sequences being equal to the common difference.

6. The control system as claimed in claim 5, the ultrasonic transducers being configured to receive the ultrasound waves, respectively, and to output respective output signals,
wherein said control system further comprises an imaging processor that is configured to be coupled to the ultrasonic transducers, and that includes
an imaging module configured to receive the output signals from each of N number of activated groups of the ultrasonic transducers, which include those of the ultrasonic transducers driven by the activating signals transmitted according to a respective one of the finite arithmetic sequences, and to process the output signals received from each of the activated groups of the ultrasonic transducers to form a respective partial-image, and
a merging module configured to merge the partial-images corresponding respectively to the activated groups to form a single ultrasonic image.

7. The control system as claimed in claim 5, wherein N is equal to or smaller than half of the number of the ultrasonic transducers.

8. The control system as claimed in claim 5, wherein said probe controller further includes a sensor module configured to detect whether said activating control module sequentially transmits two of the activating signals to sequential two of the ultrasonic transducers that are assigned with sequential two of the address parameters, and to make said activating control module inactivate the ultrasonic probe when detecting that said activating control module sequentially transmits two of the activating signals to the sequential two of the ultrasonic transducers.

9. A method for controlling an ultrasonic probe including a plurality of ultrasonic transducers that are physically arranged in a row, the method comprising the following steps of:
assigning a plurality of address parameters respectively to the ultrasonic transducers in a sequential order corresponding to a physical position order of the ultrasonic transducers in the row;
arranging the address parameters in an activation sequence, where sequential two of the address parameters in the activation sequence correspond to respective two of the ultrasonic transducers that are non-sequential to each other in the physical position order;
generating a plurality of activating signals corresponding respectively to the address parameters; and
transmitting the activating signals to the ultrasonic probe one by one every predetermined duration according to the activation sequence, each of the activating signals being transmitted to a respective one of the ultrasonic transducers that is assigned with one of the address parameters to which the activating signal corresponds so as to drive the respective one of the ultrasonic transducers to transmit and receive ultrasonic waves.

10. The method as claimed in claim 9, wherein the address parameters are integers, and are assigned respectively to the ultrasonic transducers in numerical order corresponding to the physical position order of the ultrasonic transducers in the row,
wherein the step of arranging the address parameters is to arrange one of odd ones and even ones of the address parameters in numerical order, followed by the other one of the odd ones and the even ones of the address parameters in numerical order to form the activation sequence.

11. The method as claimed in claim 10, the ultrasonic transducers being configured to receive the ultrasound waves, respectively, and to output respective output signals, wherein the method further comprises the steps of:
receiving the output signals from a first activated group of the ultrasonic transducers, which include those of the ultrasonic transducers driven by the activating signals corresponding to the odd ones of the address parameters, and processing the output signals received from the first activated group to form a first partial-image;
receiving the output signals from a second activated group of the ultrasonic transducers, which include those of the ultrasonic transducers driven by the activating signals corresponding to the even ones of the address parameters, and processing the output signals received from the second activated group of the ultrasonic transducers to form a second partial-image; and
merging the first and second partial-images to form a single ultrasonic image.

12. The method as claimed in claim 10, further comprising the steps of:
detecting whether two of the activating signals are transmitted to sequential two of the ultrasonic transducers in the physical position order; and
inactivating the ultrasonic probe when detecting that two of the activating signals are sequentially transmitted to the sequential two of the ultrasonic transducers.

13. The method as claimed in claim 9, wherein the address parameters are integers, and are assigned respectively to the ultrasonic transducers in numerical order corresponding to the physical position order of the ultrasonic transducers in the row,
wherein the step of arranging the address parameters is to arrange the address parameters into a number (N) of finite arithmetic sequences and to string the finite arithmetic sequences to form the activating sequence, where N is an integer greater than or equal to 2 and smaller than a number of the ultrasonic transducers, a difference between sequential two of the address parameters in each of the finite arithmetic sequences being equal to the common difference of N.

14. The method as claimed in claim 13, the ultrasonic transducers being configured to receive the ultrasound waves, respectively, and to output respective output signals, wherein the method further comprises the steps of:
receiving the output signals from each of multiple activated groups of the ultrasonic transducers, which include those of the ultrasonic transducers driven by the activating signals transmitted according to a respective one of the finite arithmetic sequences;
processing the output signals received from each of the activated groups of the ultrasonic transducers to form a respective partial-image; and
merging the partial-images corresponding respectively to the activated groups to form a single ultrasonic image.

15. The method as claimed in claim 13, wherein N is equal to or smaller than half of the number of the ultrasonic transducers.

16. The method as claimed in claim 13, further comprising the steps of:
detecting whether two of the activating signals are transmitted to sequential two of the ultrasonic transducers that are assigned with sequential two of the address parameters; and
inactivating the ultrasonic probe when detecting that two of the activating signals are sequentially transmitted to the sequential two of the ultrasonic transducers.
